(19) United States
(12) Patent Application Publication
(10) Pub. No.: US 2006/0111369 A1
(43) Pub. Date: May 25, 2006

(54) Synthesis, characterization and biological action of optically active isomers of floxacins

(76) Inventors: John Charin Somberg, Lake Forest, IL (US); Vasant V. Ranade, Libertyville, IL (US)

Correspondence Address: Gildo E. Fato;Laurence M. Dunlap, Ltd., Suite 202B, 14047 Petronella Drive, Libertyville, IL, 60048, US

(21) Appl. No.: 10/984,773

(22) Filed: Nov. 10, 2004

(51) Int. Cl. A61K 31/496; C07D 403/02

(52) U.S. Cl. 514/253.08; 544/363

(57) ABSTRACT

Disclosed herein is the method for synthesis and separation of enantiospecific isomers of floxacins. These isomers can be used to study the antibacterial action, as well as cardiac effects, such as arrhythmogenic activity, QT internal prolongation and dispersion, and Ikr inhibition in humans. A method for the identification of chiral isolates of the floxacins that are devoid or possess less QT prolonging action and thus cause less arthropathy especially of the Torsades de pointes variety. Also disclosed are methods for assaying these isomeric compounds present in the biological fluids.

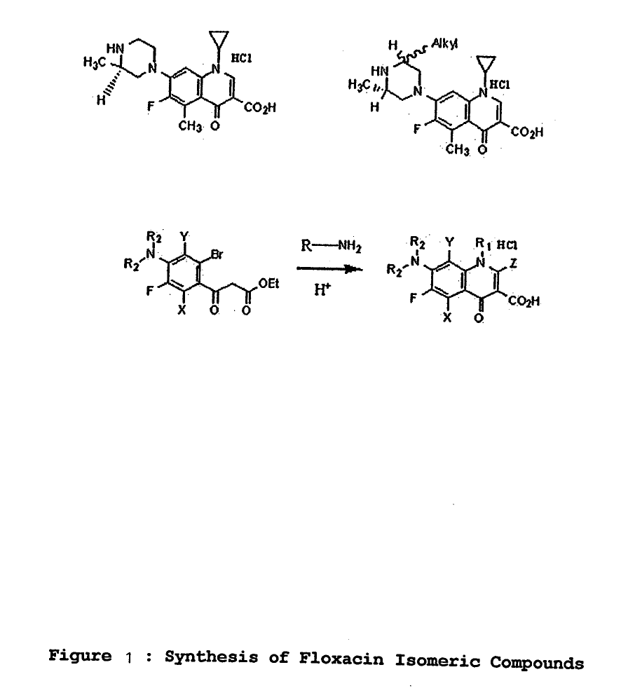

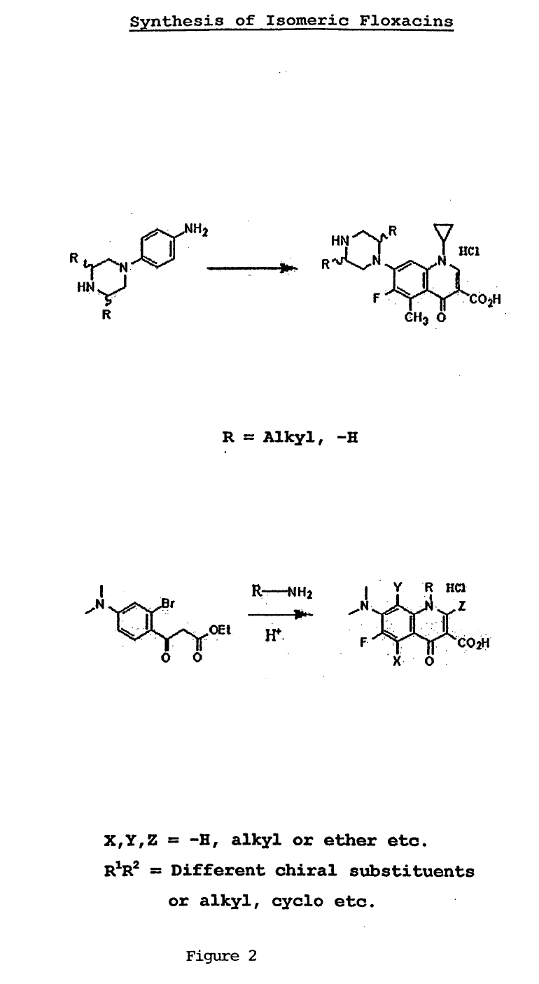

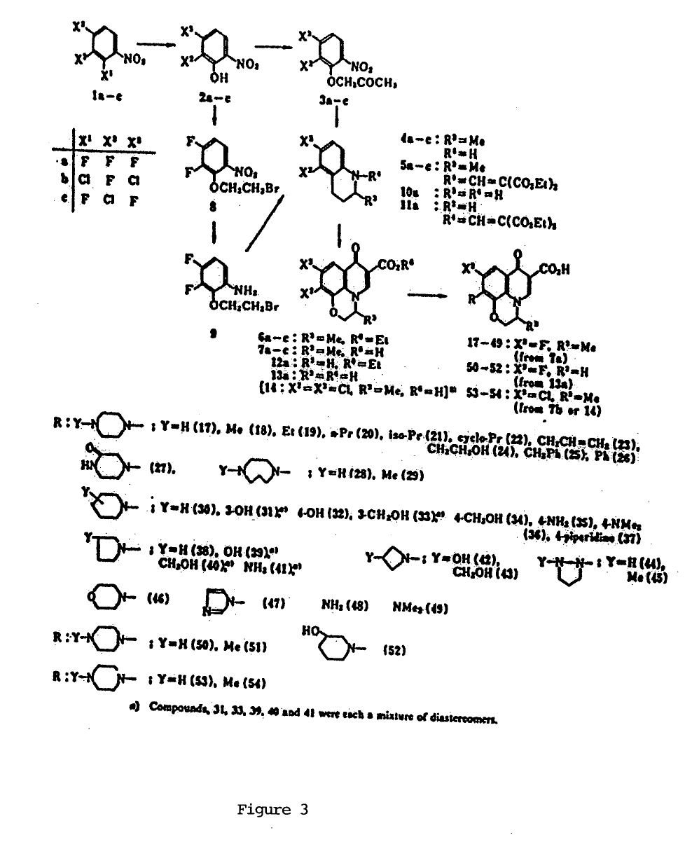

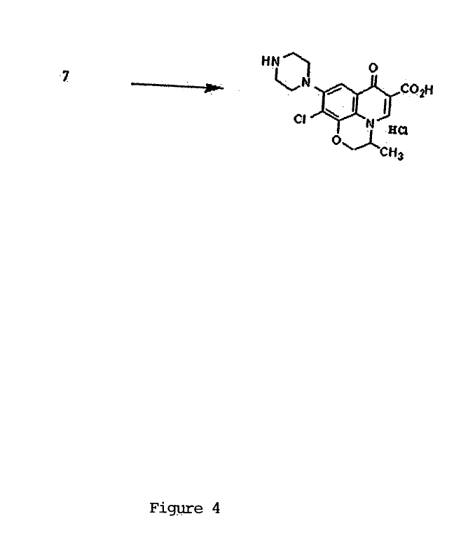

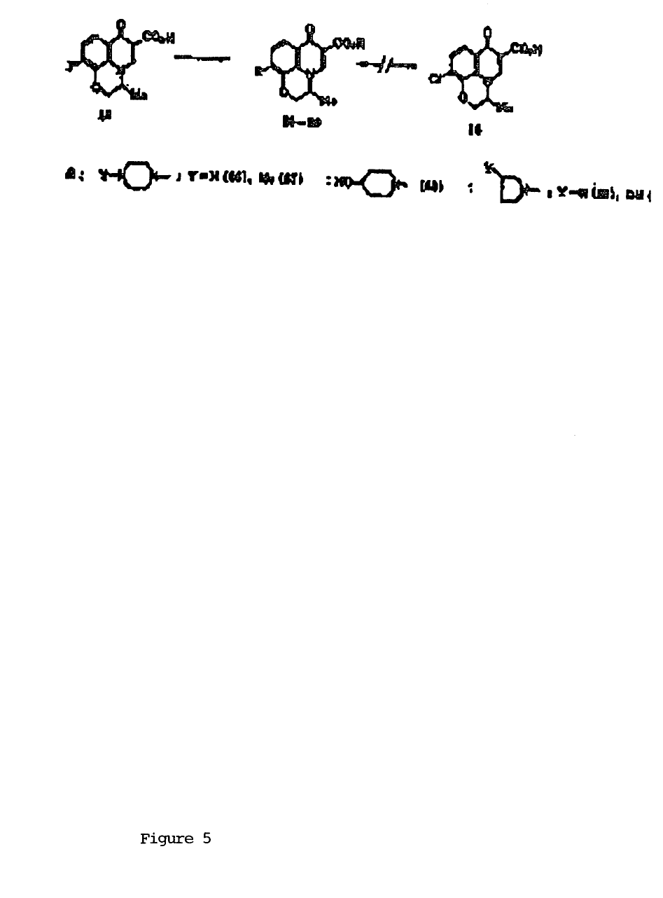

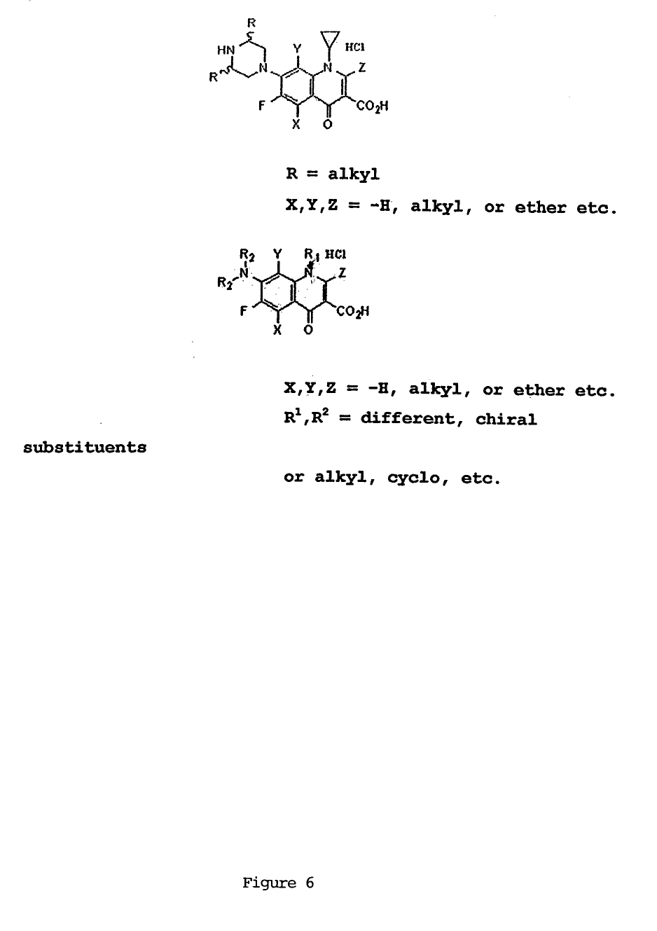

### BACKGROUND OF THE INVENTION

As antimicrobial therapy, a diverse set of antibacterial agents such as β-lactams, macrolides, tetracyclins and fluoroquinolones have been developed. In recent years, highly potent and broad-spectrum quinolone antibacterial agents, commonly known as floxacins, have been extensively studied and later clinically used for the treatment of various infections. They have been found to exhibit excellent therapeutic efficacy in the urinary tract infection, but are not always adequate for therapeutic efficacy in the respiratory tract infections. The adverse effects of quinolone antibacterial drugs have been anthralgia, joint swelling, arthropathy and arthritis and have been reported in healthy volunteers, juvenile and adult patients.

In October 1999, the FDA's Anti-Infective Drug Advisory Committee concluded that cardiac QT internal prolongation associated with moxifloxacin, a weak IKᵣ [potassium current channel blocker, should be evaluated in high risk patients. The FDA's committee recommended that it would be helpful to examine the QT effects in the subset with congestive heart failure in particular. The mean QT prolongation among moxifloxacin patients was 6 milliseconds (“msec”) with a total of 38 outliners vs. a 1 msec mean prolongation and 28 outliners in a comparator group that included a few tetracyclins. It has been known for some time that there is a dose response relationship in most patients between QT prolongation and dose (or concentration). According to the FDA, 145 cardiac events per 10,000 prescriptions have been associated with sparfloxacin. It would appear from the data that moxifloxacine may appear better than sparfloxacin but probably not quite as good as most of the other comparators. QT prolongation has been reported to be associated with an increased risk for cardiac arrhythmias, particularly the Torsades de pointes form of ventricular tachycardia.

Floxacins such as gatifloxacin, sparfloxacin, grepafloxacine and levofloxacin also prolong the QT interval. The FDA's committee and PhR & MA's joint task force have been working toward defining a preclinical threshold to serve as a predictor for the risk of drug-associated QT prolongation. They have concerns about cardiac events (arrhythmias and sudden death) and are especially concerned if the QTc intervals are increased to a duration of 50 msec or greater.

Recently, manufacturers of grepaflacin withdrew this drug from commercial sale in the U.S. Heart-rhythm abnormalities were observed in some patients during clinical trials and reports of patient deaths were reported for patients taking grepafloxacin. Chemically, grepafloxacin and other floxacins have chiral centers and therefore possess optical isomers. Recently, there has been considerable interest in preparing and testing enantiomers of drugs that exert their pharmacological action via specific receptors or enzymes. In many instances, this has been shown to result in enhanced activity, greater potency and fewer side-effects. Specifically, the anti-histamine terfenadine caused QT prolongation, while the metabolite did not. While the prokinetic agent cisapride causes QT prolongation and arrhythmias, the chiral isomer Norcisapride does not. In the case of floxacins, eg. ofloxacin, optically active enantiomers have been separated and isolated through HPLC. (−)-Ofloxacin is about 8-128 times more active as the racemate against both gram-negative and gram-positive bacteria. Subsequent biotesting has demonstrated that the S-antipode was the more active both in bacteria and in cell-free enzyme assays. Chiral isomers of the floxacin may exhibit similar or more potent antibiotic activity while exhibiting no effect or a decreased effect on the QT interval and produce less or no cardiac arrhythmias.

The use of gas chromatography in the enantiomeric separation of methamphetamine (MAP) has been extensively reviewed. Previously, differences in the pharmacological activity and pharmacokinetic behavior between enantiomers were demonstrated in the case of beta-blockers (eg. Levalbuterol and beta-amino alcohols); amphetamine (AP) methamphetamine, and penicillamine. R- and S-isomer of AP and MAP have been known for some time, with the S-isomer being approximately five times more active than the R-isomer in their effects on the CNS. Recently, similar differences have become evident in the case of antihistamine Terfenadine, the anti-depressant (fexofenadine). In the case of Fluoxetine (Prozac), a single isomer preparation is under development and in the case of Zyrtec (cetirizine), a single isomer version may also be available. The single-isomer version of cisapride (Norcisapride) has a different receptor binding profile than the parent drug. Preliminary data on the pharmacodynamics of enantiomers have indicated that one single isomer version can significantly reduce, if not eliminate, drug interaction, possess a difference in receptor binding profiles and exhibit different absorption, distribution, excretion, metabolism and toxicokinetic properties.

The results with Terfenadine indicated that the R-enantiomer of an orally administered racemate Terfenadine was preferentially oxidized in rats to form a carboxylic acid metabolite enriched in the R-enantiomer. The enantiomer is now marketed by Hoechst Marion Roussel as Allegra that has a binding profile different than the parent drug.

### SUMMARY OF THE INVENTION

The present invention proposes the development of optically active isomers of floxacins, particularly grepafloxacin, the structures of which are represented in the figures attached that possess similar or more potent antibiotic properties with a significantly reduced toxicity profile, specifically a reduced incidence of QT prolongation and thus Torsades de pointes ventricular tachycardia.

Floxacins are a class of quinolone compounds possessing antimicrobial activity. Therapeutic use of a few of these compounds was limited recently because of their adverse cardiac effects. These particular antibiotics, which are weak IKr blockers, may affect this fast potassium channel and induce arrhythmias that could be life threatening. Since floxacins possess optically active centers, separation of these isomers and testing of these compounds may permit the development of antibiotics, while not causing QT interval prolongation and therefore ventricular tachycardia of the Torsades de pointes type. The present invention also provides assays for quantitative determination of optically active isomers of floxacins in biological fluids and their antimicrobial activity.

### DETAILED DESCRIPTION OF THE INVENTION

This invention provides compounds that are optically active isomers of floxacins and their assessment of biological activity as described.

After separation, purification and characterization, each individually identified isomer is to be tested initially for in vitro anti-bacterial activity.

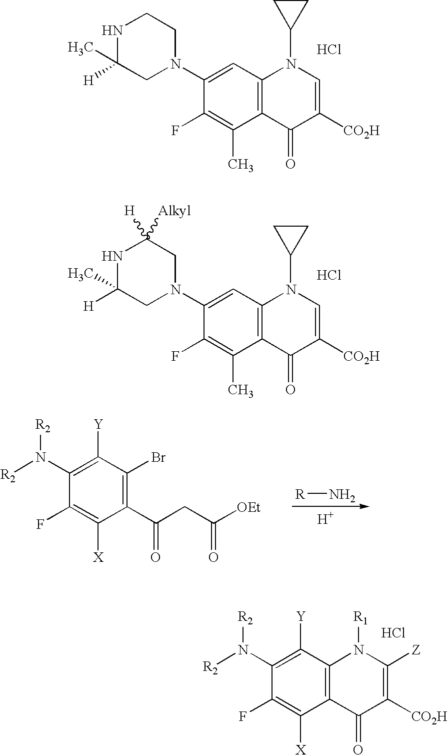

Figure: Synthesis of Floxacin Isomeric Compounds

Experimental Section

Isomer Separation Methodology:

The aim is to collect fractions of the eluent containing isomers after chromatographic separations. The chiral columns have been found to effectively separate stereoisomers and have proven to be effective tools in determining enantiomeric purity. Generally, resolution can be optimized by altering mobile phase composition and/or by selecting chiral columns with specific packing materials. Separations are performed using non-polar organic phases (eg. heptane, iso-octane) with polar organic additives such as tetrahydrofuran, alcohols, chlorinated hydrocarbons or similar solvents with or without buffer, such as phosphate or borate. Often, addition of a small amount of strong acids (eg. TFA) to the mobile phase will considerably improve separation of the isomers. Anion exchange chromatography with aqueous buffers using salt or pH gradients can also be effectively used with silica-based packings in the columns that are covalently bonded with a polymeric polyethyleneimine network that is stable in both acidic and weakly basic eluents. Alternatively, stereochemically desired floxacins can be obtained by stereospecific synthesis that is described below.

### Synthesis of Isomeric Floxacins

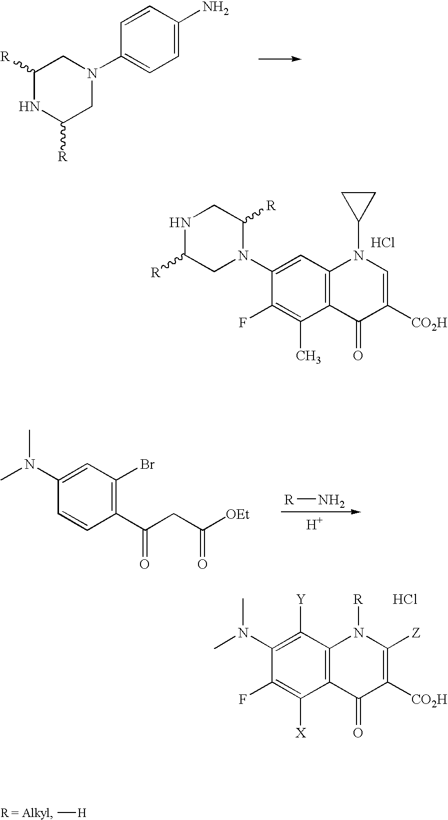

X,Y,Z=-H, alkyl or ether etc.

R¹R² =Different chiral substituents or alkyl, cyclo etc.

First, the benzoic acid derivative of the desired compound, which was a key intermediate for the synthesis of 5-methylquinolone derivatives, is prepared. Methylthiomethylation of the aniline derivative and successive treatment with activated Raney nickel gave substituted aniline derivative. Diazotization of this with sodium nitrite in concentrated HCl, followed by the treatment with KCN, afforded the nitrite. Hydrolysis of this nitrite with 50% H₂SO₄ gave the substituted methylbenzoic acid. Condensation of acid chloride with ethoxymagnesium malonate, followed by heating with p-toluenesulfonic acid in water affords methylbenzoylacetate derivative. Treatment of this with acetic anhydride and triethylorthoformate, followed by the addition of cyclopropylamine and successive cyclization with 60% NaH, gives quinolone carboxylate derivative. Hydrolysis of this with concentrated HCl in 90% AcOH afforded the corresponding acid. Finally, this acid derivative, when allowed to react with various cyclic amines (with desired stereochemical substituents) in N, N-DMF at 90° C., affords the desired floxacins.

Alternatively, commercially available substituted nitrobenzoic acid, when treated with thionyl chloride in NaOH, followed by hydrogenation with 5% Pd/c (5%), gave methyl-benzoate derivative. Regioselective dibromination of this compound with bromine and AcOH gave dibromo-methylbenzoate. Conversion of this derivative to the corresponding fluoroarene through diazonium salt can be achieved by photoirradiation in high yield. Hydrolysis of this compound with 10% NaOH gives the substituted benzoic acid derivative. The acid chloride that can be obtained by treatment with thioryl chloride on the acid derivative, on condensation with monoethyl malonate in the presence of magnesium ethoxide, affords fluorobenzoyl acetate. Final compound is obtained by carrying out the condensation reaction of piperazine derivative by heating in DMSO.

The stereochemically desired floxacins are also obtained by utilizing starting materials and condensation reaction products having suitable and desirable substituents with required stereochemical configuration.

Besides this approach, the following scheme can also be adopted for synthesis of substituted floxacins. Alkaline hydrolysis of 2,3,4-trihalogenonitrobenzenes in dimethyl sulfoxide (DMSO) occurred selectively at the halogen atom adjacent to the nitro group to give o-nitrophenol derivatives. The 2-oxopropyl ethers are converted to benzoxazine derivatives by reductive cyclization according to the method of Hill. Condensation of this compound with diethyl ethoxymethylenemalonate (EMME) by heating at 145° C. affords 5a-c. Pyridine ring cyclization of the condensates (5a-c) by heating at 145° C. in polyphosphoric ester (PPE) yielded the esters (6a-c), which are hydrolyzed with concentrated HCl in AcOH to give the corresponding acids (7a-c).

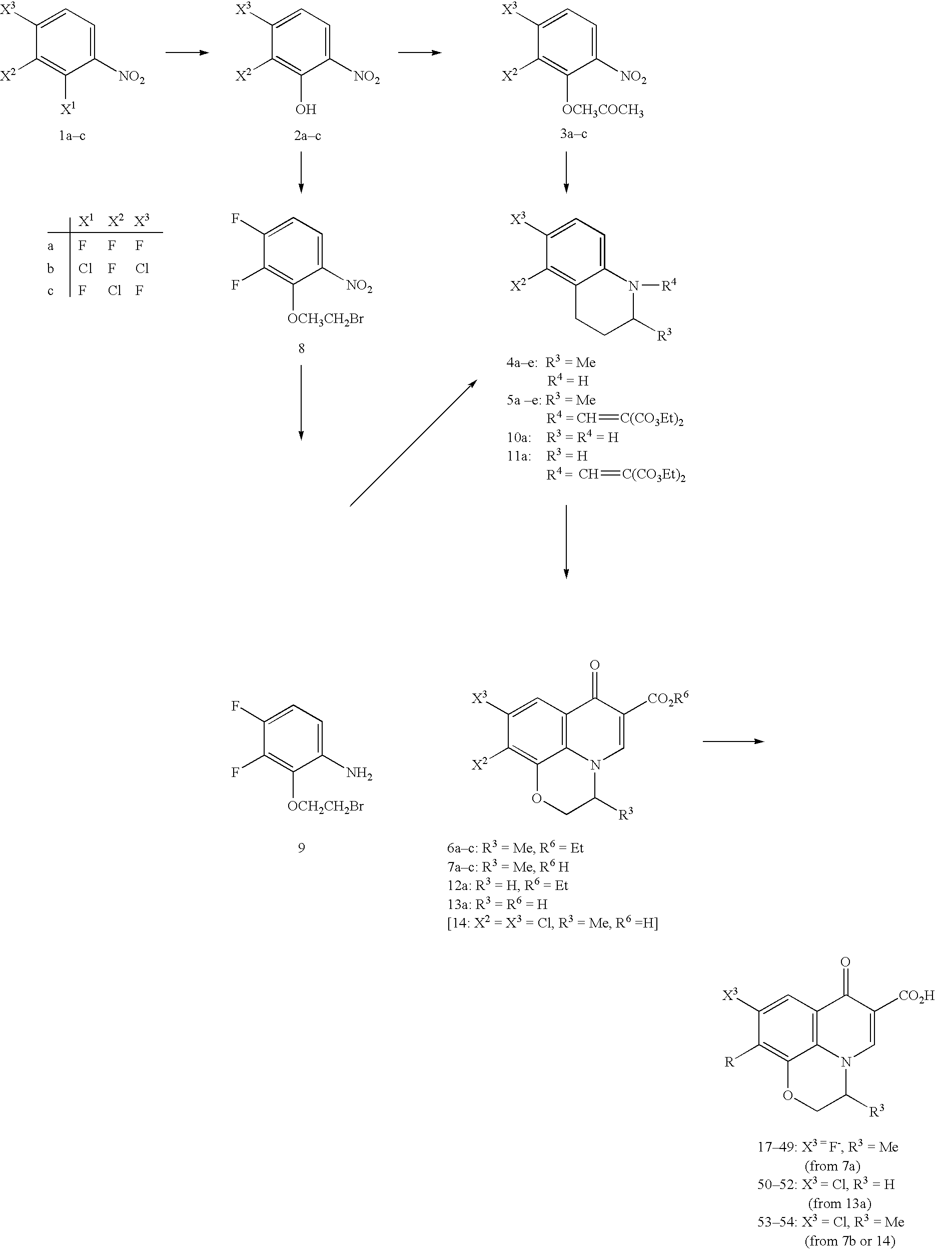

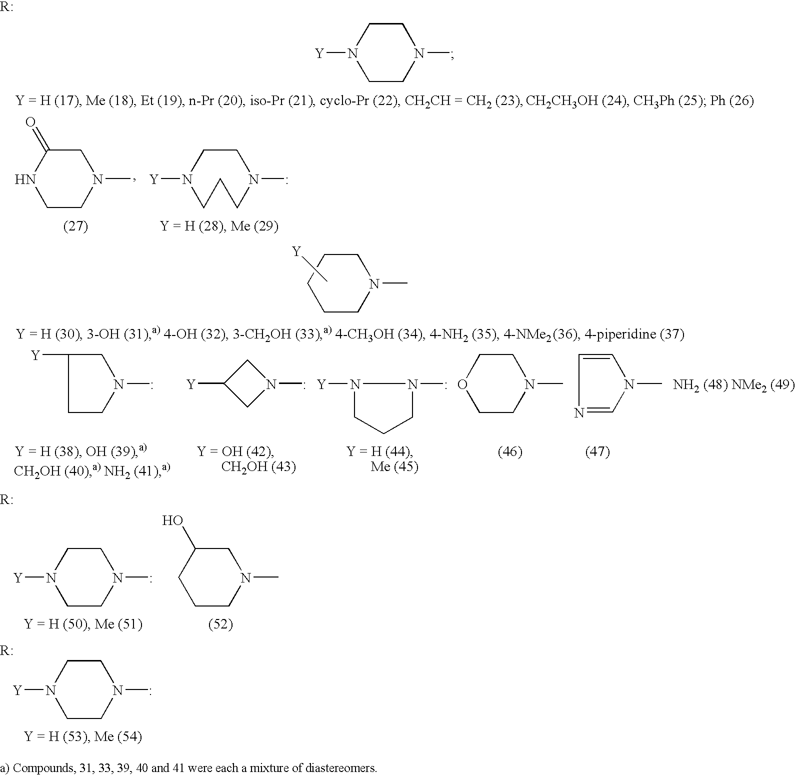

An intermediate (13) to the 3-desmethyl derivatives (50-52) is synthesized as follows: reaction of 2a with 1,2-dibromoethane in the presence of K₂CO₃ afforded compound 8, which is reduced with sodium hydrosulfite in aqueous MeOH to give the aniline derivative (9). Oxazine cyclization of 9 is carried out by heating in N,N-dimethylformamide (DMF) in the presence of K₂CO₃ to give 10. The intermediate (13) is obtained from difluoro-benzoxazine (10) in the same manner as the 3-methyl derivatives (7).

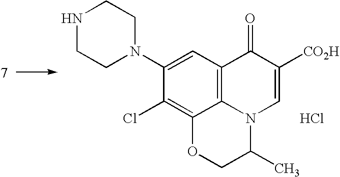

Finally, the acid (7a) is condensed with various secondary amines by heating in DMSO to afford the desired compounds (17-49) without accompanying isomer formation. 3-desmethyl derivatives are synthesized from 13 in the same manner as described for the 3-methyl derivatives. Compounds are also obtained from the 9,10-dichloro-(14) or 9-chloro-10-fluoro-(7b) derivatives. On the other hand, a 9-fluoro-10-chloro intermediate (7c) is converted to a 9-substituted 10-chloro compound (55) which shows no antimicrobial activity. 9-Dehalogenated derivatives are obtained from the 10-fluoro derivative (15) by heating with amines, while the 10-chloro derivative (16) did not react with amines under the same conditions. 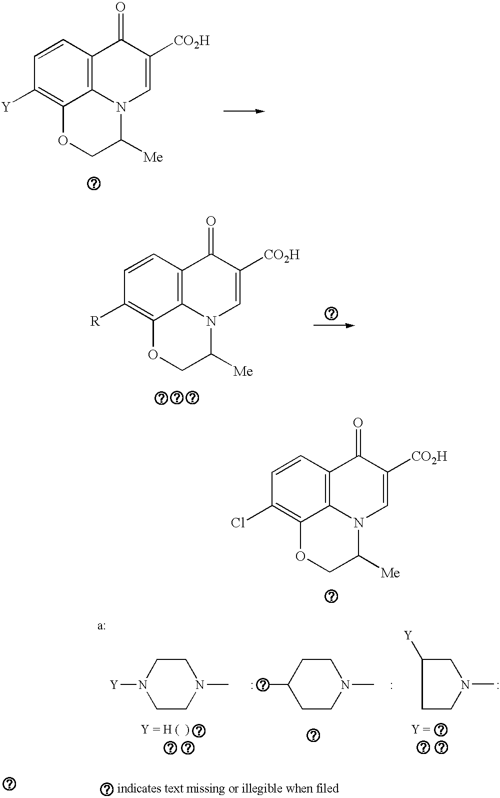
Assay of Floxacins from Biological Fluids
Serum and Urine Collection

Blood sufficient to provide 1.5 mL of serum for determination of floxacin concentrations is collected from each subject before dosing and at the following times afterwards: 0.25, 0.5, 0.75, 1, 1.5, 2, 3, 4, 6, 8, 12, 18, 24, 36, 48, 60 and 72 hours. The whole blood samples are allowed to clot for 45 minutes at room temperature and then spun in a refrigerated centrifuge. The harvested serum samples are immediately frozen at −29° C. until analysis.

Urine samples are obtained from all subjects before dosing and at the following intervals after dosing: 0-2, 2-4, 4-8, 8-12, 12-24, 24-48 and 48-72 hours. At the end of each collection interval, the total urine volume for the interval is measured and after thorough mixing, a 10 mL sample from each collection period is stored at −70° C. until analysis.

Analysis of Floxacin in Serum and Urine

The concentrations of floxacin in serum and urine are determined by a reverse-phase high performance liquid chromatography (HPLC) method with UV detection, as reported previously (Teng et al., 1993a). Following solid-phase extraction, chromatographic separation is carried out using a C₁₈ column and a phosphate mobile phase. Floxacin and the internal standard (a methyl derivative floxacin) are detected by ultraviolet absorbance at 275 nm. The calibration curves are linear over a concentration range 0.1-20.0 mL/L (R²=0.999). The average recoveries are over 70% for both compounds.

Serum Protein Binding

Serum protein binding of floxacin is examined by equilibrium dialysis at nominal predialysis floxacin concentrations of 1 and 5 mg/L. Fresh blank serum was obtained from fasting healthy volunteers. Dialysis is performed in 1.36 mL dialysis cell blocks (Spectra/Por Equilibrium Dialyzer) separated with Spectra/Por membranes (12,000-14,000 mol. Wt. Cutoff). The membranes are prepared by sequentially soaking,in HPLC grade water, 30% ethanol and 0.1 M phosphate buffer (pH 7.4) for 30, 60 and 30 minutes, respectively. The dialysis cells are immersed in a water bath at 37° C. and rotated on a dialysis wheel at 20 rpm. The time to reach equilibrium was previously determined to be 6 hours. At the end of dialysis, aliquots from both the serum and buffer are collected for determination of floxacin concentration using the HPLC-UV method. Each serum sample evaluated is dialyzed in duplicate, with post-dialysis buffer and serum samples being determined in triplicate. The percentage of free floxacin in serum is estimated, with adjustment for osmotically induced volume shifts across the membrane.

Protein Binding

Protein binding in serum is determined at different concentrations between 0.3 and 2.1 mg/liter by the micropartition MPS-1 system for separation of free from protein-bonded solutes (Amicon GmbH, Witten, Germany). Separation is done at 22° C., and incubations are performed at 37° C.

High-Performance Liquid Chromatography

Ofloxacin and the metabolites demethyl-ofloxacin and ofloxacin-N-oxide are determined by high-performance liquid chromatography. Basically, the method consists of reverse-phase chromatography and fluorimetric detection (6). The stationary phase is Nucleosil 5C18 (Macherey & Nagel, Duren, Germany), employed at room temperature. The detector (Schoeffel FS 970; Kratos, Inc., Karlsruhe, Federal Republic of Germany) is operated under the following conditions: excitation, 295 nm; emission cutoff, 418 nm; sensitivity range, 0.05 to 0.20 μA. Fluorescence based yields of the demethyl and N-oxide metabolites are 0.95 and 0.77 relative to ofloxacin. Detection limits of the method are 0.22 mg/liter in serum and 0.2 mg/liter in urine for ofloxacin and its two metabolites. Precision from day to day (coefficient of variation) ranges from 2.2 to 5.2% (serum) and 2.9 to 3.3% (urine) for ofloxacin. For the metabolites, precision within series is 7.2% (ofloxacin N-oxide) and 3.6% (demethyl ofloxacin). The mobile phase consisted of 86 parts (vol/vol) of a solution of tetrabutyl ammonium phosphate, 1.0 mmol/liter, adjusted to pH 2 and 14 parts of acetonitrile. For improved separation of the demethyl metabolite, the pH was adjusted to 5.

Pharmacokinetic Analysis

The pharmacokinetic analyses after oral dosing of the substance are based on an open two-compartment model, corresponding to the following equation (2, 12, 16), C9t)=Ae^{-α(t-to)}+Be^{-β(t-to)}−C(t)ₒe^{-ka(t-to)}, where C(t) represents the concentration in serum at time t in milligrams per liter, A and B (in milligrams per liter) are the zero intercepts of the tangents α and β with the ordinate, and α and β (per minute) represent the slopes of the rapid initial and slow terminal distribution and elimination phases, respectively; Kₐ is the absorption rate, and tₒ is the lag time. After i.v. administration, open two- and three-compartment models were used, following the equations: Ae^{-α(t)}+Be^{-β(t)} (open two-compartment model) and Ae^{-α(t)}+Be^{-β(t)}+Ce^{-r(t)}, where C(t) represents the serum concentration at time t in milligrams per liter, A, B, and C (in milligrams per liter) are the zero intercepts of the tangents α, β, and γ with the ordinate, and α and β (per minute) represent the slopes of the rapid initial, intermediate, and slow terminal distribution and elimination phases, respectively. The secondary pharmacokinetic parameters were calculated with the aid of these hybrid constants (A, B, and C and α, β, and γ). The least-squares is used to fit the regression curve to the experimentally obtained values of the serum concentration curve (after normalization of the serum concentrations to a mean body weight of 70.0 kg). The pharmacokinetic parameters (for each test subject individually) are calculated by the method of nonlinear regression analysis using a program developed for pharmacokinetic applications. The duration of infusion is treated by the method of Loo and Riegelmann.

The mathematical calculation of the constants and pharmacokinetic parameters is performed by standard methods as previously described. The Wilcoxon test for paired differences is used to distinguish the differences within the pharmacokinetic parameters.

Biology Activity

In Vitro Antibacterial Activity

Minimum inhibitory concentration (MIC) is determined by the two-fold agar dilution method with Muller-Hinton agar (Difco Laboratories, Detroit, Mich. U.S.A.). The overnight broth cultures are diluted to approximately 10⁶ CFU mL⁻¹ with fresh broth, and an inoculum of 10⁻⁴ CFU per spot is applied to agar plates containing graded concentrations of each compound with an incubating apparatus. After incubation at 37° C. for 18 hours, the MIC is defined as the minimum drug concentration which inhibited the growth of bacteria.

In Vivo Antibacterial Activity

In vivo activity is determined against the experimental systemic infections caused by gram-positive and gram-negative pathogens. ICR strain mice (male, 20-25 g) are divided into groups of 10.staph.aureus and E. coli no. 29 are preincubated in nutrient broth and S.pneumoniae type III is preincubated in brain heart infusion broth containing 5% of horse serum for 18 hours at 37° C. The bacteria is suspended in the same fresh media and mucin is added before injection. Mice are infected intraperitonally with 0.5 mL of the respective pathogens. One hour following injection of the bacteria, a single dose of each compound is administered orally to mice. The survival rate on day seven is calculated and the ED₅₀ value is determined by the probit method.

The following optically active isomeric compounds of floxacins are to be separated for biological evaluation using the methods described. These separation methods and the outcome of the results may not be limited in their scope. 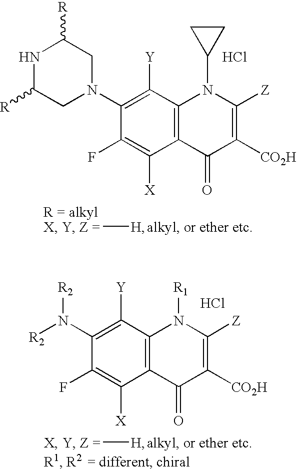
substituents
or alkyl, cyclo, etc.

Optically active isomeric compounds of the formula (see above) will be tested for

a. In vitro antibacterial activity

b. In vivo antibacterial activity

c. QT internal/dispersion IKr inhibition studies

These tests will be carried out in animals and humans using established procedures (see also experimental section).

AV Node Contractility Curve Determination:

The guinea pig or rat will be anesthetized with Pentobarbital at 40 mg/kg, i.p. The animal is connected to an EKG recorder after anesthesia. Then the animal is placed on a small animal respirator. Aseptic surgical instruments are soaked in ethanol and then flamed. An incision is made in the chest wall to open the chest. A Walton Brody stain gauge arch is sutured to the right ventricle to measure contractile changes. The refractoriness of the AV node is determined by measuring the PR interval on the surface EKG. Graphs can be created from the data displaying the dose related change in PR interval to the dose related contractile augmentation. Floxacin chiral isolates will be infused through the jugular vein at a number of concentrations (from 0.1 μM to 1 μM and then to 10 μM), at a constant rate by infusion pump, to determine the differential effects on the AV node and cardiac contractility. The infusions are continued to a 50% augmentation in contractility or the development of ventricular tachycardia. To maintain a consistent level of anesthesia, pentobarbital 20 mg/Kg IP or 10 mg/Kg IV for more immediate action at 30-minute intervals will be given. The study will first be performed in 15 of both guinea pigs and rats, and select the optimum specie (either guinea pig or rat) for further study. Then floxacin isomers will be tested separately in each group of 15 animals to assure consistency of results. After the completion of the experiment, the animals receive a fatal intravenous dose of anesthesia.

Molecular Pharmacology Studies of HERG Expressed in Xenopus Oocytes:

Xenopus Oocytes Isolation

The female Xenopus Laevis frogs (Nasco, Modesto, Calif., U.S.A.) were anesthetized with 0.2% tricaine methanesulfonate (Sigma, St. Louis, Mo., U.S.A.). The ovarian lobes were surgically removed and digested with 2.0 mg/ml type IA collagenase (Sigma) for 2 hours in a Ca²⁺-free solution containing (mmol/L): 88.0 NaCl, 1.0 KCl, 2.4 NaHCO₃, 5.0 HEPES, 0.82 MgCl₂ solution (pH 7.6 with NaOH) to remove the follicle layer. At the end of collagenase treatment, the stage V and VI oocytes were picked up and stored in modified Barth's solution containing (mmol/L): 88.0 NaCl, 1.0 KCl, 2.4 NaHCO₃, 5.0 HEPES, 0.30 Ca(NO₃)₂, 0.40 CaCl₂, 0.82 MgSO₄, 2.5 pyruvic acid and gentamicin 50 μg/ml, pH 7.6 with NaOH.

Expression of HERG in Oocytes

The BERG clone was a gift from Dr. Gail Robertson (University of Wisconsin, Madison, Wis.), and the cDNA was cloned into the pGH19 vector. The cDNA was lineralized with NOT1 and in vitro transcription was made with T7 mMessage Machine Kit (Ambion, Austin, Tex., U.S.A.). One day after isolation, oocytes were injected 40 nl of 50 μg/μl of HERG cRNA using microinjector (Drummond Scientific, Broomall, Pa., U.S.A.) and incubated at 18-20° C. in the modified Barth's solution.

Electrophysiology

Currents were studied 2-7 days after injection by two microelectrodes voltage clamp technique at room temperature (21-23°). The oocytes were voltage-clamped with an amplifier (Warner OC-725 C oocyte clamp, Warner Instruments, Hamden, Conn.). Current and voltage electrodes were filled with 3 mol/L KCl and had a resistance of 2-4 MΩ for voltage-recording electrodes and 1-2 MΩ for current-passing electrodes. The recording bath solution contained in mmol/L: 96 NaCl, 5.0 KCl, 2.0 CaCl₂, 1.0 MgCl₂, 5 HEPES; and the pH of solution was adjusted to 7.4 with NaOH. In some experiments, 2.5 KCl and 10.0 KCl was applied instead of 5.0 KCl to test the extracellular potassium concentration effect on quinidine blockage. Recordings were made before and after 5 minutes of the drug perfusion. Stock solution of floxacins (50 mM) were made up in dimethylsulphoxide (DMSO) and kept at −20° C. Appropriate drug dilution with 5K recording solution was prepared shortly before the experiments. The portion of DMSO in the perfusion solutions did not exceed 0.2% to avoid artificial effects. Data acquisition was made with pCLAMP software (Axon Instruments, Foster City, Calif., U.S.A.). Experiments in which the holding current was more than 200 nA at −80 mV holding potential were excluded from analysis. Statistical significance between the data is obtained by Student's t-test or by the ANOVA test. When appropriate, data are expressed as mean±SD.

What is claimed is:

1. Optically active isomers of floxacins selected from the group consisting of:
a) grepafloxacin-l
b) grepafloxacin-d
c) moxifloxacin-l
d) moxifloxacin-d
e) levofloxacin-l
f) levofloxacin-d
g) sparfloxacin-l
h) sparfloxacin-d
i) ofloxacin-l
j) ofloxacin-d
k) gatifloxacin-l
l) gatifloxacin-d
m) lomefloxacin-l
n) lomefloxacin-d
o) temafloxacin-l
p) temafloxacin-d
q) trovafloxacin-l
r) trovafloxacin-d

2. An optically active isomer of floxacin as claimed in claim 1 comprising, e.g. grepafloxacin-l.

3. An opically active isomer of floxacin as claimed in claim 1 comprising, e.g. grepafloxacin-d.

4. A process for preparing a selected stereochemically desired floxacine, said process comprising of treating a selected substituted nitrobenzoic acid with thionyl chloride in NaOH followed by hydrogenation with 5% Pd/c to produce the methyl benzoate derivative; regioselective dibromination of the methyl benzoate derivative with bromine and AcOH to produce dibromo methylbenzoate; photoirradiation of the dibromo methylbenzoate with diazomium salt to produce the corresponding fluoroarene; hydrolysis of the fluoroarene with 10% NaOR to provide the selected substituted benzoic acid derivative; treating the benzoic acid derivative with thioryl chloride and conducting a condensation reaction with monoethyl malonate in the presence of magnesium ethoxide to produce fluorobenzoyl acetate; and obtaining the selected stereochemically desired floxacin by conducting a condensation reaction of the acetate by heating in dimethylsulfoxide.

7. Chiral isolates of the floxacins that possess equal or greater antibacterial activity to the parent compound(?) but less prolongation of the QT internal.

8. Chiral isolate of the floxacins that possess equal or greater antibacterial activity to the parent racemate compound but less or no QT dispersion.

9. Chiral isolate of the floxacins that possess equal or greater antibacterial activity to the parent racemate compound but less or no inhibition of the Ikr potassium chiral.

10. Chiral isolate of the floxacins that possess equal or greater antibacterial activity to the parent compounds but cause less or no risk of cardiac arrhythmias.

11. A floxacin chiral isolate with less antibacterial activity but still with a superior therapeutic toxic ratio than the parent floxacin racematic compound.
